(19) United States
(12) Patent Application Publication
(10) Pub. No.: US 2004/0009130 A1
(43) Pub. Date: Jan. 15, 2004

(54) Compositions for treating keratinous surfaces

(76) Inventors: Donna Marie Detore, Morris Plains, NJ (US); Gale McElroy Reinhart, Middletown, NJ (US); James Joseph Ferone, Bridgewater, NJ (US)

Correspondence Address: Revlon Consumer Products Corporation, 625 Madison Avenue, New York, NY, 10022, US

(21) Appl. No.: 10/190,804

(22) Filed: Jul. 8, 2002

(51) Int. Cl. A61K 7/42; A61K 7/06; A61K 35/78

(52) U.S. Cl. 424/59; 424/757; 424/74

(57) ABSTRACT

A cosmetic composition comprising a mixture of extracts from yam and soy in combination with at least one protective agent which is a daytime protective agent, a night time protective agent, or mixtures thereof and methods for ameliorating the adverse effects of aging and menopause on skin.

### TECHNICAL FIELD

The invention is in the field of compositions for treatment of keratinous surfaces such as skin (including lips), hair and nails.

### BACKGROUND OF THE INVENTION

Treatment of skin with various types of botanical extracts is well known. As women age, cosmetics companies are becoming more interested in incorporating botanical extracts into skin treatment products because, in many cases, these extracts are believed to contain very minute amounts of ingredients that exert a beneficial effect on the skin. When women approach menopausal age, certain skin conditions become more noticeable such as laxity, skin roughness, and dryness. It is believed that these skin conditions and the other effects experienced by women are due to a decrease in the levels of the hormone estrogen. Many women alleviate the uncomfortable symptoms of estrogen loss by taking oral or topically absorbed estrogen in the form of pills, patches, or creams. However, there are some known negative effects from use of such preparations. In particular, it has been said that estrogen supplementation during menopause may increase the risk of cancer.

It has long been known that extracts from certain plants contain molecules that are very similar to estrogen in chemical structure. These estrogen-like compounds are referred to as “phytoestrogens”. Phytoestrogens are believed to have some minimal estrogenic activity at very high concentrations. Proponents of this theory cite the well known fact that women from far eastern countries that have plant based diets consisting of soy based products (which have high levels of phytoestrogens) do not generally exhibit menopausal symptoms of the severity found in western women. This is believed to be due to the significant amounts of phytoestrogens consumed by these women in their daily diet.

For years health food stores have been selling lotions and creams containing extract of Dioscorea Villosa (yam) with the claim that such preparations alleviate the symptoms associated with estrogen loss. Soy based products, primarily in the orally ingestible form, are also well known dietary supplements. For years both botanists and practicioners of holistic medicine have believed that products from both the soy and yam plants, such as extracts and oils, have value in the treatment of various female ailments including those associated with menopause. However, the various types of skin creams and lotions containing yam extract that are available in health food stores are not very aesthetically pleasing, nor are such products formulated in such a manner as to enhance the penetration of the extracts into the skin. Further, the soy based products that are available are found only in the ingestible form.

Accordingly, there is a need for aesthetically pleasing skin care products containing ingredients derived from soy and yam that have maximum efficacy and are able to better penetrate the skin. The method by which the skin care product will exhibit maximum efficacy and skin penetration depends on whether the composition is used during the daytime or night time hours.

One problem with such products during day time use is that while the products contain ingredients that will exert beneficial effects on the skin, the efficacy of the beneficial ingredients in the extracts are compromised by contact with the elements. For example, when the consumer applies a skin cream containing the beneficial ingredients to the skin and spends time outdoors, the wind, sun, or rain will immediately reduce the effect of the extract on the skin. The result is that the consumer doesn't obtain the full benefit of the beneficial ingredients on the skin because they are either washed off or inactivated on the skin by contact with the elements. In the same way, night creams with the beneficial extract are applied to the skin, usually prior to retiring. The cream is rubbed off the facial skin while the consumer sleeps, which in turn reduces the beneficial effects of the ingredients on the skin.

It is an object of the invention to provide cosmetic compositions for application to skin containing extracts from soy and yam in combination.

It is a further object of the invention to maximize the penetration of the combination of soy and yam extracts into the skin by formulating a cosmetic composition with ingredients that better retain the combination on the skin.

It is a further object of the invention to maximize the efficacy of the combination of soy and yam extract by reducing the tendency of the extract to physically degrade or be removed from the skin surface by various elemental conditions.

### SUMMARY OF THE INVENTION

The invention is directed to a cosmetic composition comprising a mixture of extracts derived from soy and yam in combination with at least one protective agent which is a daytime protective agent, a night time protective agent, or mixtures thereof.

The invention is also directed to a method for enhancing the efficacy of a combination of extracts from soy and yam in skin care compositions comprising adding to said composition at least one protective agent which is a daytime protective agent, a night time protective agent, or mixtures thereof.

### DETAILED DESCRIPTION

The term “keratinous surfaces” means the surfaces of skin, hair and nails. The term “skin” when used herein is in the broad sense meaning the skin of the face, body, and neck as well as the lips.

The compositions of the invention may be anhydrous, or in the emulsion form. If the latter, the emulsions may be water-in-oil or oil-in-water. Suitable water-in-oil emulsions contain about 0.1-95%, preferably about 0.5-85%, more preferably about 5-85% by weight of the total composition of water and about 0. 1-99%, preferably about 1-90%, more preferably about 3-85% by weight of the total composition of oil. The term “protective agent” means an ingredient or combination of ingredients that maximizes the effectiveness of the extract combination on the skin by either promoting better penetration of the extracts into the skin, better retaining the extract on the skin, or prevent the degradation of the extracts on the skin.

A. The Yam Extract

The yam extract used in the claimed compositions is preferably obtained from the genus Dioscorea, including Dioscorea Villosa and the like. The extract may be derived from the root, flower, or leaves but is preferably obtained from the yam root. The extract may be present ranging from about 0.0001-25%, preferably about 0.0005-20%, more preferably about 0.001-18% by weight of the total composition. Suitable yam extract is available from a variety of sources including Sederma. The extract may be present in an aqueous solution or suspension containing other ingredients in addition to water, such as glycols, etc.

B. The Soy Extract

Preferably, the soy extract used in the claimed compositions is obtained from soybeans. The extract may be obtained from the actual bean, or the associated flowers or roots. The material extracted from the soybean may be in the form of a protein, a peptide, carbohydrate, ester. Particularly preferred is where the extract is obtained from the soybean itself, and is in the form of an oil. Most preferred is where the extract is a sterol obtained from the bean, also referred to as soybean sterols. The soybean derived material is present ranging from about 0.0001-25%, preferably about 0.0005-20%, more preferably about 0.001-18% by weight of the total composition

C. The Day Time Protective Agent

The term “day time protective agent” means an agent which enhances the efficacy of the combination of soy and yam extracts when the composition used by a consumer who is engaging in typical day time activities. The day time protective agent may enhance the efficacy of the extracts by synergistically interacting with the extract components to enhance the UV activity of the sunscreens. The combination of the extracts and the day time protective agent may also enhance the efficacy of the composition in the amelioration of the effects of wind and rain on the skin, or causing the composition containing this combination to exhibit improved effects in ameliorating lines, wrinkles, skin imperfections, laxity, skin tone, age spots, skin discoloration, and the other negative effects of estrogen loss (for example as found in menopause and peri-menopause) on the skin.

The day time protective agent includes a chemical or physical sunscreen. Suitable day time protective agents include UVA and UVB chemical sunscreens and/or physical sunscreens.

1. UVA Chemical Sunscreens

The day time protective agent preferably comprises at least one UVA sunscreen. The term “UVA sunscreen” means a chemical compound that blocks UV radiation in the wavelength range of about 320 to 400 nm. Preferred UVA sunscreens are dibenzoylmethane compounds having the general formula:

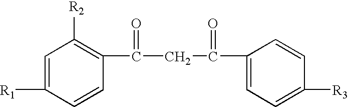

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4diisopropylbenzoylmethane, 4-tert-butyl-4′-methoxydibenzoylmethane, 4,4′-diisopropylbenzoylmethane, 2-methyl-5-isorpoyl-4′-methoxydibenzoymethane, 2-metyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

The claimed compositions may contain from about 0.001-20%, preferably 0.005-5%, more preferably about 0.005-3% by weight of the composition of UVA sunscreen. In the preferred embodiment of the invention the UVA sunscreen is Avobenzone, and it is present at not greater than about 3% by weight of the total composition.

2. UVB Chemical Sunscreens

The term “UVB sunscreen” means a compound that blocks UV radiation in the wavelength range of from about 290 to 320 nm. A variety of UVB chemical sunscreens exist including α-cyano-β,β-diphenyl acrylic acid esters as set forth in U.S. Pat. No. 3,215,724, which is hereby incorporated by reference in its entirety. One particular example of a α-cyano-β,β-diphenyl acrylic acid ester is Octocrylene, which is 2-ethylhexyl 2-cyano-3,3-diphenylacrylate. In certain cases the composition may contain no more than about 10% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

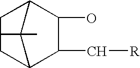

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

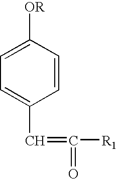

wherein R and R₁ are each independently a C₁₋₂₀ straight or branched chain alkyl. Preferred is where R is methyl and R₁ is a branched chain C₁₋₂₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as the UVB screening agents are various benzophenone derivatives having the general formula:

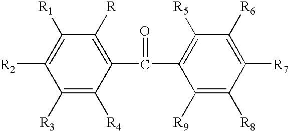

R through R₉ are each independently H, OH, NaO₃S, SO3H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone) and Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

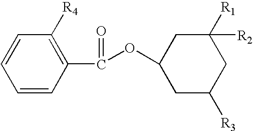

wherein R₁, R₂, R₃, and R4 are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

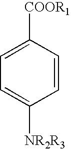

Wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula:

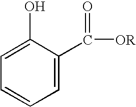

wherein R is a straight or branched chain alkyl, including derivatives of the above compound formed from mono-, di-, or triethanolamines. Particular preferred are octyl salicylate, TEA-salicylcate, DEA-salicylate, and mixtures thereof.

Generally, the amount of the UVB chemical sunscreen present may range from about 0.001-45%, preferably 0.005-40%, more preferably about 0.01-35% by weight of the total composition.

3. Physical Sunscreens

The day time protective agent may also include one or more physical sunscreens. The term “physical sunscreen” means a material that is generally particulate in form that is able to block UV rays by forming an actual physical block on the skin. Examples of particulates that serve as solid physical sunblocks include titanium dioxide, zinc oxide and the like in particle sizes ranging from about 0.001-150 microns.

In general, it is preferred that the claimed compositions contain SPF (sun protective factor) values ranging from about 1-30, preferably about 2-20, most preferably about 3-18. Calculation of SPF values is well known in the art. Preferably, the claimed compositions have SPF values greater than 4.

D. The Night Time Protective Agent

Suitable night time protective agents that facilitate efficacy of the combination of soy and yam extracts on the skin by either promoting absorption of the extract into the skin by contributing to form a substantive layer of the cosmetic composition containing the extracts onto the skin. The substantive layer keeps the active ingredients, including the extract, on the skin surface and facilitates absorption of such actives into the skin or retention of such actives on the skin surface so they can better perform their intended function. Such ingredients may also be referred to as skin penetration enhancers in that they promote penetration of actives into the skin due to the substantive effect they provide on the skin. Examples of such materials include silicone oils, semi-solid or solid waxy materials, triglycerides, esters, paraffinic hydrocarbons, and the like. Preferably the night time protective agent is found in the claimed composition in ranges of from about 0.1-75%, preferably about 0.5-50%, more preferably about 1-40% by weight of the total composition. Such night time protective agents include those further described herein.

1. Silicone Oils

Particularly suitable as the protective agent are various types of silicone oils including water soluble or water insoluble volatile or non-volatile silicone oils. The term “volatile” means that the silicone has a measureable vapor pressure, i.e. a vapor pressure of at least 2 mm. of mercury at 20° C. If volatile, the silicone generally will have a viscosity of about 0.5 to 25 centistokes at 25° C. Suitable volatile silicones include cyclic silicones, linear silicones, or mixtures thereof. Cyclic silicones (or cyclomethicones) are of the general formula:

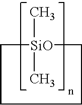

where n=3-6.

Linear volatile silicones in accordance with the invention have the general formula:

(CH₃)₃Si—O—[Si(CH₃)₂—O]ₙ—Si(CH₃)₃

where n=0-6, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, and mixtures thereof.

The silicone may also be nonvolatile, and in particular water insoluble nonvolatile silicones. The term “nonvolatile” means that the silicone has a vapor pressure of less than 2 mm. of mercury at 20° C. A variety of silicones fit this definition including dimethicone, phenyl trimethicone, diphenyl dimethicone, hexadecyl methicone, stearoxydimethicone, stearyl dimethicone, cetyl dimethicone, and so on. Preferred is where the nonvolatile silicone, if present, has a viscosity ranging from about 5-1,000,000 centistokes at 20° C., more preferably about 50-500,000 centistokes at 20° C.

2. Esters

Suitable protective agents also include various types of esters. In general such esters have the formula RCO—OR wherein each R is independently a C₁₋₂₅ straight or branched chain saturated or unsaturated alkyl, alkylcarbonyloxyalkyl, or alkoxycarbonylalkyl, aryl, which may be substituted or unsubstituted with halogen, hydroxyl, alkyl, and the like.

Examples of suitable esters include alkyl acetates, alkyl behenates, alkyl lactates, alkyl benzoates, alkyl octanoates, alkyl salicylates, and in particular C₁₂₋₁₅ alkyl benzoate. Examples of further esters are set forth on pages 1670-1676 of the CTFA International Cosmetic Ingredient Handbook, Eighth Edition, 2000, which is hereby incorporated by reference.

3. Fats and Oils

Fats and oils are also suitable as protective agents. They may be further defined as glyceryl esters of fatty acids (triglycerides), as well as the synthetically prepared esters of glycerin and fatty acids having the following general formula:

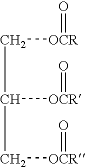

wherein R, R′, and R″ are each independently fatty acid radicals. Examples of such materials include oils such as apricot kernel oil, avocado oil, canola oil, olive oil, sesame oil, peanut oil, trilinolenin, trilinolein, trioctanoin, tristearin, triolein, sesame oil, rapeseed oil, sunflower seed oil, and so on.

4. Fatty Acids

Fatty acids may also serve as protective agents. Fatty acids are the carboxylic acids obtained by hydrolysis of animal or vegetable fats and oils. Carboxylic acids having alkyl chains shorter than about seven carbon atoms are not generally considered fatty acids. Fatty acids have the general structure R—COOH where R is a straight or branched chain saturated or unsaturated C₇₋₆₅ alkyl. Examples of suitable fatty acids include arachidic acid, arachidonic acid, behenic acid, capric acid, caproic acid, caprylic acid, coconut acid, corn acid, cottonseed acid, hydrogenated coconut acid, hydroxystearic acid, lauric acid, linoleic acid, linolenic acid, linseed acid, myristic acid, oleic acid, palmitic acid, palm kernel acid, soy acid, tallow acid, and the like.

5. Fatty Alcohols

Suitable fatty alcohols include those made by reducing the fatty acid —COOH group to the hydroxyl function. They generally have the formula RCH₂OH. Examples of fatty alcohols are behenyl alcohol, C₉₋₁₁ alcohol, C₁₂₋₁₃ alcohol, C₁₂₋₁₅ alcohol, C₁₂₋₁₆ alcohol, caprylic alcohol, cetearyl alcohol, cetyl alcohol, coconut alcohol, decyl alcohol, lauryl alcohol, myristyl alcohol, oleyl alcohol, and the like.

6. Hydrocarbons

Hydrocarbons also serve as good skin penetrants for use as the night time protective agent. Hydrocarbons are generally chemically inert. Examples of suitable hydrocarbons include C₇₋₆₀ isoparaffins, ethane, heptane, hexane, hydrogenated polyisobutene, isobutane, isododecane, isoeicosane, isohexadecane, isopentane, microcrystalline wax, mineral oil, mineral spirits, paraffin, petrolatum, petroleum distillates, squalene, polyethylene, and mixtures thereof. Preferred hydrocarbons are mineral oil and polyethylene.

Preferred is where the night time protective agents are silicone oil, fatty alcohols, esters, fatty acids, and mixtures thereof.

E. Other Ingredients

The claimed composition may contain other ingredients in addition to the combination of soy and yam extracts, day time protective agent, and night time protective agents. Such ingredients include other botanical extracts, humectants, preservatives, polymers, particulates, surfactants, and the like.

1. Organic Surfactants

Preferably, the claimed compositions contain one or more surfactants including those having anionic, nonionic, amphoteric, zwitterionic, or cationic moieties. The organic surfactant, if present, should range from about 0.001-30%, preferably about 0.005-25%, preferably about 0.01-20% by weight of the total composition. The compositions of the invention preferably comprise about 0.01-20%, preferably about 0.1-15%, more preferably about 0.5-10% by weight of the total composition of a surfactant. Particularly preferred are one or more nonionic surfactants or emulsifiers including alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol.

Also suitable as the nonionic surfactant are alkyoxylated carboxylic acids, which are formed by the reaction of a carboxylic acid with an alkylene oxide or with a polymeric ether. The resulting products have the general formula:

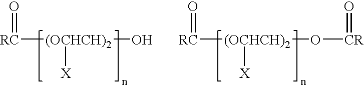

where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula:

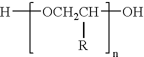

wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

Also suitable as nonionic surfactants are silicone surfactants, which are defined as silicone polymers which have at least one hydrophilic radical and at least one lipophilic radical. The silicone surfactants may be a liquid or solid at room temperature and are generally of the water-in-oil or oil-in-water type. Preferably, they have an Hydrophile/Lipophile Balance (HLB) of 2 to 18. Preferably the organosiloxane is a nonionic surfactant having an HLB of 2 to 12, preferably 2 to 10, most preferably 4 to 6. The HLB of a nonionic surfactant is the balance between the hydrophilic and lipophilic portions of the surfactant and is calculated according to the following formula:

HLB=7+11.7χlog M_{w}/M_{O}

where M_{w} is the molecular weight of the hydrophilic group portion and Mₒ is the molecular weight of the lipophilic group portion.

The term “organosiloxane polymer” means a polymer containing a polymeric backbone including repeating siloxy units that may have cyclic, linear or branched repeating units, e.g. di(lower)alkylsiloxy units, preferably dimethylsiloxy units. The hydrophilic portion of the organosiloxane is generally achieved by substitution onto the polymeric backbone of a radical that confers hydrophilic properties to a portion of the molecule. The hydrophilic radical may be substituted on a terminus of the polymeric organosiloxane, or on any one or more repeating units of the polymer. In general, the repeating dimethylsiloxy units of modified polydimethylsiloxane emulsifiers are lipophilic in nature due to the methyl groups, and confer lipophilicity to the molecule. In addition, longer chain alkyl radicals, hydroxy-polypropyleneoxy radicals, or other types of lipophilic radicals may be substituted onto the siloxy backbone to confer further lipophilicity and organocompatibility. If the lipophilic portion of the molecule is due in whole or part to a specific radical, this lipophilic radical may be substituted on a terminus of the organosilicone polymer, or on any one or more repeating units of the polymer. It should also be understood that the organosiloxane polymer in accordance with the invention should have at least one hydrophilic portion and one lipophilic portion.

The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof.

The term “lipophilic radical” means an organic radical that, when substituted onto the organosiloxane polymer backbone, confers lipophilic properties to the substituted portion of the polymer. Examples of organic radicals which will conver lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof. The C₁₋₄₀ alkyl may be non-interrupted, or interruped by one or more oxygen atoms, a benzene ring, amides, esters, or other functional groups.

The polymeric organosiloxane surfactant used in the invention may have any of the following general formulas:

MₓQ_{y}, or

MₓT_{y}, or

MDₓD′_{y}D″_{z}M

wherein each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof. T is a trifunctional siloxy unit having the empirical formula RR′SiO_{1.5} or RRSiO_{1.5}. Q is a quadrifunctional siloxy unit having the empirical formula SiO₂, and D, D′, D″, x, y, and z are as set forth below, with the proviso that the compound contains at least one hydrophilic radical and at least one lipophilic radical. Preferred is a linear silicone of the formula:

MDₓD′_{y}D″_{z}M

wherein M=RRRSiO_{½}

D and D′=RR′SiO_{2/2}

D″=RRSiO_{2/2}

x, y, and z are each independently 0-1000,

where R is methyl or hydrogen, and R′ is a hydrophilic radical or a lipophilic radical, with the proviso that the compound contains at least one hydrophilic radical and at least one lipophilic radical.

Most preferred is wherein

M=trimethylsiloxy

D=Si[(CH₃)][(CH₂)ₙCH₃]O_{2/2} where n=0-40,

D′=Si [(CH₃)][(CH₂)ₒ—O—PE)]O_{2/2} where PE is (—C₂H₄O)ₐ(—C₃H₆O)_{b}H, o=0-40,

a=1-100 and b=1-100, and

D″=Si(CH₃)₂O_{2/2}

More specifically, suitable silicone surfactants have the formula:

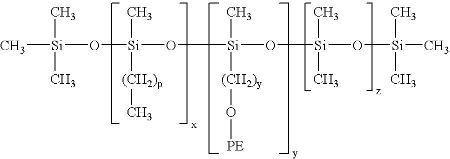

wherein p is 0-40, and

PE is (—C₂H₄O)ₐ(—C₃H₆O)_{b}—H

where x, y, z, a, and b are such that the maximum molecular weight of the polymer is approximately about 50,000.

Another type of preferred organosiloxane emulsifier suitable for use in the compositions of the invention are emulsifiers sold by Union Carbide under the Silwet™ trademark. These emulsifiers are represented by the following generic formulas:

(Me₃Si)_{y-2}[(OSiMe₂)_{x/y}O—PE]_{y}

wherein PE=-(EO)ₘ(PO)ₙR

R=lower alkyl or hydrogen

Me=methyl

EO is polyethyleneoxy

PO is polypropyleneoxy

m and n are each independently 1-5000

x and y are each independently 0-5000, and

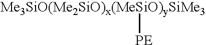

wherein PE=—CH₂CH₂CH₂O(EO)ₘ(PO)ₙZ

Z=lower alkyl or hydrogen, and

Me, m, n, x, y, EO and PO are as described above,

with the proviso that the molecule contains a lipophilic portion and a hydrophilic portion.

Also suitable as nonionic silicone surfactants are hydroxy-substituted silicones such as dimethiconol, which is defined as a dimethyl silicone substituted with terminal hydroxy groups.

Examples of silicone surfactants are those sold by Dow Corning under the tradename Dow Corning 3225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention.

Suitable cationic, anionic, zwitterionic, and amphoteric surfactants are disclosed in U.S. Pat. No. 5,534,265, which is hereby incorporated by reference in its entirety.

2. Humectants

Preferably the compositions of the invention comprise 0.01-30%, preferably 0.5-25%, more preferably 1-20% by weight of the total composition of one or more humectants. Suitable humectants include materials such as glycols, sugars, and the like. Suitable glycols include polyethylene and polypropylene glycols such as PEG 4-240, which are polyethylene glycols having from 4 to 240 repeating ethylene oxide units; as well as C₁₋₆ alkylene glycols such as propylene glycol, butylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆, preferably C₂₋₄ alkylene glycols, most particularly butylene glycol.

3. Other Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, farnesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2. Preferred are botanical extracts including Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, and mixtures thereof.

4. Gellants

It may be desireable to include other gellants in the oil or water phase of the composition to provide gelling. Such gellants may be included a range of about 0.1-20%, preferably about 1-18%, more preferably about 2-10% by weight of the total composition is suggested. Suitable gellants include soaps, i.e. salts of water insoluble fatty acids with various bases. Examples of soaps include the aluminum, calcium, magnesium, potassium, sodium, or zinc salts of C₆₋₃₀, preferably C₁₀₋₂₂ fatty acids.

Also suitable are hydrocolloids such as gellan gum, gum arabic, carrageenan, and those set forth in U.S. Pat. No. 6,197,319 which is hereby incorporated by reference in its entirety.

Water soluble synthetic polymeric materials are also good gellants, including polymers of acrylic acid or C₁₋₂₀ esters thereof, which may be crosslinked or uncrosslinked. Examples include Carbopol (polymer of acrylic acid crosslinked with a polyfunctional agent which is the allyl ether of sucrose or the allyl ether of pentaerythritol), and the like.

5. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

6. Particulates

It may be desirable to incorporate one or more particulate materials such as pigments, powders, and the like into the claimed composition to provide various effects. For example, inclusion of small amounts of powders will improve consistency of the composition and may provide a more aesthetic color. If desired, small amounts of pigments may also be included, particularly in the event where it is desired to provide color to the skin. If present the particulates range from about 0.001-20%, more preferably about 0.01-18% of particulate matter having a particle size of 0.01 to 200, preferably 0.25-100 microns. The particulate matter may be colored or non-colored (for example white) non-pigmentitious powders. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

The particulate matter component also may comprise various organic and/or inorganic pigments. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

The claimed compositions provide an excellent vehicle for the Soy/yam extract, and enhance the efficacy and other beneficial properties of the extract on the skin. The compositions may be found in a variety of forms including skin lotions and creams, lipsticks, lip balms, foundation makeup, blush, eyeshadow, toners, astringents and the like.

Suitable lipsticks or lip balms generally include pigments in combination with oils, structuring agents (waxes or other polymeric materials that add viscosity). Suitable foundation makeup compositions are found in the emulsion form or can be anhydrous.

The invention will be further described in connection with the following examples which are set forth for the purposes of illustration only.

### EXAMPLE 1

An oil-in-water emulsion cream suitable for day time wear was prepared as follows:

| | w/w |
|---|---|
| INGREDIENT | % |
| Butylene glycol | 5.0 |
| Preservatives | 1.73 |
| Magnesium Ascorbyl Phosphate | 0.01 |
| Silica | 0.75 |
| Glycerin | 5.0 |
| Talc | 0.75 |
| Carbomer (2.5% aqueous solution) | 20.0 |
| Octyl methoxycinnamate | 7.5 |
| Octyl salicylate | 3.0 |
| Homosalate | 5.0 |
| Benzophenone-3 (Oxybenzone) | 2.0 |
| 4-tert-butyl methoxydibenzoylmethane (Avobenzone) | 2.0 |
| Dimethicone | 2.0 |
| Cetyl Alcohol | 1.5 |
| Stearyl Alcohol | 0.75 |
| Talc | 0.75 |
| PPG-2 Myristyl Ether Propionate | 4.5 |
| C12-15 Alkyl Benzoate | 1.0 |
| Tocopheryl Acetate | 0.1 |
| Aloe Barbadensis Leaf Extract | 0.1 |
| Retinyl Palmitate | 0.01 |
| Lauryl Lactate | 1.5 |
| Butylene Glycol Dicaprylate/Dicaprate | 5.0 |
| Peg 100 Stearate | 0.75 |
| Polysorbate 60 | 2.6 |
| Sorbitan stearate | 0.9 |
| Triethanolamine | 1.0 |
| Mica, Titanium | 1.0 |
| Glycyrrhia Glabra extract in cyclomethicone | 1.0 |
| Salix Nigra (willowbark) Extract | 1.0 |
| Oleyl alcohol, Dioscorea Villosa (Yam) Root Extract, Glycine | 1.0 |
| Sojo (soybean) sterols | |
| Trifolium Pratense (Clover) Flower Extract, glycerin, butylene | 1.0 |
| glycol, lecithin | |
| Water, glycerin, Macrocystis Pyrifera Extract, hydrolyzed wheat | 1.0 |
| protein | |
| PEG-40 hydrogenated castor oil, Pyrus Malus (apple) Fruit extract | 0.5 |
| Saxifraga Sarmentosa Extract, Vitis Vinifera (grape) Fruit Extract, | 0.5 |
| butylene glycol, Morus Bombycis (Mulberry) Root extract, | |
| Scutellaria Baicalensis Root extract, disodium EDTA, water | |
| Methoxypropylgluconamide | 0.3 |
| Sodium hydroxide | 0.050 |
| Kinetin | 0.05 |
| Anthemis Nobilis Flower Extract (chamomile Roman), Salvia | 0.3 |
| Sclarea (clary) extract, citrus medica limonum (lemon) peel | |
| extract | |
| Water | QS |

The composition was prepared by heating water, glycols, preservatives, magnesium ascorbyl phosphate, silica, glycerin and talc to 80° C. with sweep mixing. Once uniform, the carbomer solution was added with sweep agitation maintaining a temperature of 80° C. In a separate vessel oil phase ingredients (Octyl methoxycinnamate to Sorbitan stearate) were mixed with propeller agitation and heated to 80° C. Once both phases reached 80° C. the oil phase was transferred into the water phase with fast agitation. Once the transfer was completed the composition was neutralized with triethanolamine, followed by addition of the mica and titanium dioxide. The mixture was homogenized for 15 minutes, then cooled to 50° C. with continuous agitation. Once the bulk was cooled to 50° C. the remaining ingredients were added with mixing. The sodium hydroxide and kinetin were premixed with water (1%) in a container before adding to the mixture. The mixture was then cooled to 30° C. and poured into suitable containers.

### EXAMPLE 2

An oil-in-water emulsion face cream suitable for night wear was prepared as follows:

| | w/w |
|---|---|
| INGREDIENT | % |
| Preservatives | 0.65 |
| Beeswax | 1.25 |
| Hydrogenated polyisobutene | 5.00 |
| Sorbitan stearate | 3.00 |
| Hydrogenated coco-glycerides | 1.00 |
| Octyldodecanol | 2.00 |
| Cetearyl ethylhexanoate | 3.00 |
| Capric caprylic triglycerides | 3.00 |
| Glyceryl stearate | 2.00 |
| Cetearyl alcohol, ceteareth-20 | 3.00 |
| Stearic acid | 3.15 |
| Tetradibutyl Pentaeryrityl hydroxyhydrocinnamate | 0.05 |
| Glycerrhiza Glabra (licorice) Extract in cyclomethicone | 1.00 |
| Cyclomethicone | 1.00 |
| Triethanolamine | 0.79 |
| Phenoxyethanol | 1.00 |
| Oleyl alcohol, Dioscorea Villosa (wild yam) Root Extract, Glycine | 1.00 |
| Soja (soybean) sterols | |
| Water | QS |

The cream was prepared by heating the water, preservatives, and magnesium ascorbyl phosphate, glycerin and glycols to 80° C. with sweep mixing. Once uniform, the carbomer solution was added with sweep agitation maintaining the temperature at 80° C. In a separate vessel the oil phase ingredients (dimethicone through glyceryl stearate, PEG 100 stearate in the above formula) were mixed with propeller agitation and heated to 80° C. Once both phases reached 80° C. the oil phase was transferred into the water phase with fast agitation. Once transfer was completed the mixture was neutralized with triethanolamine, followed by homogenization for 15 minutes. The mixture was cooled to 60° C. with continuous agitation, after which the water and sclerotium gum mixture was added to the mixture and further homogenized for an additional 15 minutes. Then bulk was then cooled to 50° C. and glyceryl polyacrylate, dimethiconol, and cyclomethicone were added to the batch, followed by further homogenization for 15 minutes. Then the remaining ingredients were added with mixing. Sodium hydroxide, kinetin, and water (1%) were premixed in a container, then added to the mixture. The mixture was then cooled to 30° C. and poured into suitable containers.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A cosmetic composition comprising a mixture of extracts from soy and yam in combination with at least one protective agent which is a daytime protective agent, a night time protective agent, or mixtures thereof.

2. The composition of claim 1 wherein the yam extract is from the genus Dioscorea.

3. The composition of claim 2 wherein the yam extract is obtained from the root of a yam plant of the genus and species Dioscorea Villosa.

4. The composition of claim 1 the soy extract is obtained from the soybean and is a protein, peptide, carbohydrate, or oil.

5. The composition of claim 4 wherein the soybean extract is an oil which is a sterol.

6. The composition of claim 1 which is in the emulsion form.

7. The composition of claim 6 which is a water-in-oil emulsion.

8. The composition of claim 6 which is an oil-in-water emulsion.

9. The composition of claim 1 wherein the daytime protective agent comprises a chemical sunscreen or a mixture of a chemical sunscreen and a physical sunscreen, or mixtures thereof.

10. The composition of claim 9 wherein the chemical sunscreen is a UVA sunscreen, a UVB sunscreen, or mixtures thereof.

11. The composition of claim 10 wherein the chemical sunscreen is a mixture of UVA and UVB sunscreens.

12. The composition of claim 1 wherein the daytime protective agent is a mixture of UVA and UVB sunscreens and the composition has an SPF of at least about 4.

13. The composition of claim 12 wherein the UVA sunscreen is dibenzoyl methane compound.

14. The composition of claim 12 wherein the UVB sunscreen is α an-cyano-β,β-diphenyl acrylic acid ester, a benzylidene camphor compound, a cinnamate compound, a benzophenone compound, a menthyl salicylate compound, a benzoic acid derivative, a salicylate compound, or mixtures thereof.

15. The composition of claim 14 wherein the UVB sunscreen is Cinoxate, Homosalate, methyl anthranilate, Octocrylene, octyl methoxycinnamate, octyl salicylate, Oxybenzone, Padimate O, phenylbenzimidazole sulfonic acid, Sulisobenzone, TEA-salicylate, and mixtures thereof.

16. The composition of claim 9 wherein the physical sunscreen is titanium dioxide, zinc oxide, or mixtures thereof.

17. The composition of claim 13 wherein the dibenzoylmethane compound is Avobenzone, and it is present at no more than about 3% by weight of the total composition.

18. The composition of claim 15 wherein the Cinoxate is present at no more than about 3% by weight of the total composition.

19. The composition of claim 15 wherein the Homosalate is present at no more than about 15% by weight of the total composition.

20. The composition of claim 15 wherein the Octocrylene is present at no more than about 10% by weight of the composition.

21. The composition of claim 15 wherein the padimate O is present at no more than about 8% by weight of the total composition.

22. The composition of claim 1 which is in the emulsion form and comprises at least one daytime protective agent which is a mixture of UVA and UVB sunscreens.

23. The composition of claim 22 wherein the emulsion is an oil-in-water emulsion.

24. The composition of claim 22 wherein the UVA sunscreen is Avobenzone.

25. The composition of claim 22 wherein the UVB sunscreen is Cinoxate, Homosalate, Octocrylene, octyl methoxycinnamate, octyl salicylate, Oxybenzone, Padimate O, or mixtures thereof.

26. The composition of claim 22 additionally containing one or more plant extracts selected from the group consisting of Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, and mixtures thereof.

27. The composition of claim 1 wherein the night time protective agent is a silicone oil, triglyceride, ester, fatty alcohol, fatty acid, hydrocarbon, or mixtures thereof.

28. The composition of claim 27 wherein the silicone oil comprises cyclomethicone, dimethicone, phenyl trimethicone, or mixtures thereof.

29. The composition of claim 28 wherein the night time protective agent is a fatty alcohol, fatty acid, or mixtures thereof.

30. The composition of claim 1 further comprising one or more humectants.

31. The composition of claim 1 further comprising one or more thickening agents.

32. The composition of claim 1 further comprising one or more nonionic surfactants.

33. A method for enhancing the efficacy of a combination of extracts from soy and yam in the amelioration of skin lines, wrinkles, imperfections, laxity, skin tone, age spots, and/or skin discoloration, comprising adding to said composition a protective agent which is a day time protective agent, a night time protective agent, or mixtures thereof.

34. The method of claim 33 wherein the day time protective agent comprises a sunscreen.

35. The method of claim 33 wherein the night time protective agent comprises a silicone.

36. A method for ameliorating the adverse effects of estrogen loss on skin pursuant to menopause or peri-menopause, comprising treating the skin with a composition containing a mixture of extracts from soy and yam in combination with at least one protective agent which is a daytime protective agent, a night time protective agent, or mixtures thereof.

37. The method of claim 36 wherein the adverse effects of estrogen loss on skin pursuant to menopause or peri-menopause include skin dryness, roughness, laxity, discoloration, or age spots.

38. The method of claim 37 wherein the protective agent is a day time protective agent and the composition is applied to the skin in the form of a day cream or lipstick.

39. The method of claim 37 wherein the protective agent is a night time protective agent and the composition is applied to the skin in the form of a night cream.

40. A method for improving skin tone comprising treating the skin with a composition containing a mixture of extracts from soy and yam in combination with at least one protective agent which is a daytime protective agent, a night time protective agent, or mixtures thereof.

41. The method of claim 40 wherein the composition contains a daytime protective agent and comprises a day cream or lipstick.

42. The method of claim 40 wherein the composition contains a night time protective agent and comprises a night cream.
